(19) United States
(12) Patent Application Publication
(10) Pub. No.: US 2018/0182496 A1
(43) Pub. Date: Jun. 28, 2018

(54) INFORMATION PROCESSING APPARATUS, SYSTEM, INFORMATION PROCESSING METHOD, AND MEDIUM

(71) Applicant: Canon Kabushiki Kaisha, Tokyo (JP)

(72) Inventors: Masahiro Yakami, Kyoto-shi (JP); Yutaka Emoto, Kyoto-shi (JP); Naoki Matsuki, Tokyo (JP)

(21) Appl. No.: 15/845,827

(22) Filed: Dec. 18, 2017

(30) Foreign Application Priority Data
Dec. 28, 2016 (JP) 2016-256152

(51) Int. Cl. G16H 50/70; G16H 15/00; G16H 50/20; G16H 10/60; G16H 30/20; G06F 17/30

(52) U.S. Cl. CPC G16H 50/70; G16H 15/00; G16H 50/20; G06F 17/30528; G16H 30/20; G06F 17/30554; G06F 17/30598; G16H 10/60

(57) ABSTRACT

An apparatus that searches for suitable similar cases is provided. The apparatus includes an identification unit that identifies, in a case where a first diagnosis item including a description about a diagnosis, a second diagnosis item including a description about a diagnosis different from the first diagnosis item, and link information indicating a relationship between the first diagnosis item and the second diagnosis item are acquired as a search key, a diagnosis report including the first diagnosis item and the second diagnosis item and in which the link information is added, from among a plurality of stored diagnosis reports.

### BACKGROUND

### Field

The present disclosure relates to information processing apparatuses, systems, information processing methods, and programs.

### Description of the Related Art

When a search for similar cases is conducted, it is desirable to refine a search result using the description of a radiological interpretation report being generated so that cases needed to generate the radiological interpretation report are efficiently acquired.

Japanese Patent Application Laid-Open No. 2015-197738 discusses a method of refining a search result by searching for similar cases using, as a search condition, a set of a cause that is a start of drug administration and an effect that is a drop in blood pressure.

There are demands for searching for appropriate cases using a relationship, e.g., relationship between diagnoses, between items of the same category, such as a diagnosis and a finding, even in a case of a radiological interpretation report that includes items of a plurality of categories instead of a relationship between items of different categories, such as a cause and an effect.

### SUMMARY

Aspects of the present disclosure are directed to, but not limited to, providing an apparatus that searches for an appropriate case.

According to an aspect of the present disclosure, an information processing apparatus configured to output a specific report from among a plurality of diagnosis reports includes an identification unit configured to identify, in a case where a first diagnosis item including a description about a diagnosis, a second diagnosis item including a description about a diagnosis that is different from the first diagnosis item, and link information indicating a relationship between the first diagnosis item and the second diagnosis item are acquired as a search key, a diagnosis report that includes the first diagnosis item and the second diagnosis item and in which the link information is added, from among a plurality of stored diagnosis reports, and an output unit configured to output the identified diagnosis report.

According to another aspect of the present disclosure, an information processing apparatus includes a transmission unit configured to transmit, as a search key, a first diagnosis item including a description about a diagnosis, a second diagnosis item including a description about a diagnosis that is different from the first diagnosis item, and link information indicating a relationship between the first diagnosis item and the second diagnosis item, and a reception unit configured to receive, as a search result based on the search key, a diagnosis report that includes the first diagnosis item and the second diagnosis item and in which link information is added.

Further features will become apparent from the following description of exemplary embodiments with reference to the attached drawings.

### BRIEF DESCRIPTION OF THE DRAWINGS

FIG. 1 illustrates an example of the system configuration of a medical report generation apparatus.

FIG. 2 illustrates an example of the configuration of an entire medical information system.

FIG. 3 illustrates an example of a conceptual diagram of a medical document to be generated.

FIGS. 4A and 4B illustrate a storage example of entry information and link information.

FIG. 5 illustrates an example of the functional configuration of a medical document generation apparatus.

FIGS. 6A and 6B illustrate an example of a method of automatically adding a relationship between entries.

FIG. 7 illustrates an example of a graphical user interface (GUI) of a medical document generation apparatus.

FIGS. 8A, 8B, 8C, and 8D illustrate an example of a GUI for inputting a search condition of a case search.

FIGS. 9A and 9B are a flowchart illustrating a process of a case search.

FIG. 10 illustrates an example of a method of presenting a case search result.

FIG. 11 is a flowchart illustrating an example of a process of a case search according to a second exemplary embodiment.

FIG. 12 illustrates an example of a search condition of a case search according to the second exemplary embodiment.

FIG. 13 illustrates an example of cases having a time-series relationship.

### DESCRIPTION OF THE EMBODIMENTS

Examples of exemplary embodiments will be described below with reference to the attached drawings. Configurations described in the below-described exemplary embodiments are mere examples, and the present disclosure is not limited to the illustrated configurations.

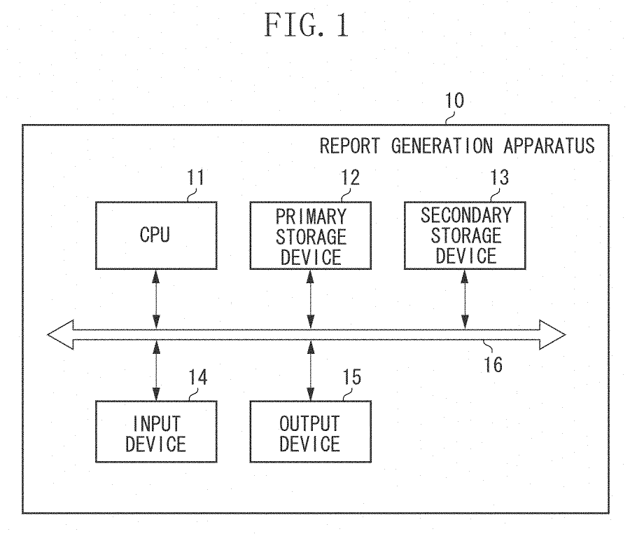

A first exemplary embodiment will be described below. FIG. 1 schematically illustrates a system configuration that realizes a report generation apparatus. A report generation apparatus 10 illustrated in FIG. 1 corresponds to an example of an information processing apparatus.

The report generation apparatus 10 generates a radiological interpretation report (hereinafter, also referred to simply as “report”) based on user input. The report generation apparatus 10 searches for radiological interpretation reports corresponding to a search condition from a radiological interpretation report database 2. While the foregoing describes that the report generation apparatus generates a radiological interpretation report and conducts a search for a radiological interpretation report, an apparatus that generates a radiological interpretation report and an apparatus that conducts a search for a radiological interpretation report can be separate apparatuses.

The report generation apparatus 10 includes a central processing unit (CPU) 11, a primary storage device 12, a secondary storage device 13, an input device 14, an output device 15, and a system bus 16. The CPU 11, the primary storage device 12, the secondary storage device 13, the input device 14, and the output device 15 included in the report generation apparatus 10 are communicably connected to one another via the system bus 16.

The CPU 11 controls operations of the entire system and executes programs stored in the primary storage device 12. The CPU 11 can be a single CPU or a plurality of CPUs. The programs can be executed by a graphics processing unit (GPU), field-programmable gate array (FPGA), or application-specific integrated circuit (ASIC). The type of a processor that executes the programs is not particularly limited. The processor can be a plurality of processors.

The primary storage device 12 is mainly a memory such as a random-access memory (RAM) and reads and stores programs stored in the secondary storage device 13.

The secondary storage device 13 is, for example, a recording medium such as a hard disk or flash memory. In general, the capacity of the primary storage device 12 is smaller than the capacity of the secondary storage device 13, and programs, data, etc. that cannot be stored in the primary storage device 12 are stored in a secondary storage device. Data and the like that need to be stored for a long time are also stored in the secondary storage device. In the present exemplary embodiment, a program for realizing a process of the exemplary embodiment is stored in the secondary storage device 13, and at the time of executing the program, the CPU 11 load the program into the primary storage device 12 and executes the program.

The input device 14 is, for example, a device that inputs information to the report generation apparatus 10. The input device 14 includes one or more of a pointing device, such as a mouse, a touch panel, a keyboard, etc.

The output device 15 is a device that outputs information to an external device. The output device 15 includes a monitor such as a liquid crystal display (LCD). A combination of any number of monitors can be used as the output device 15.

The system bus 16 is a bus configured to communicably connect the components included in the report generation apparatus 10.

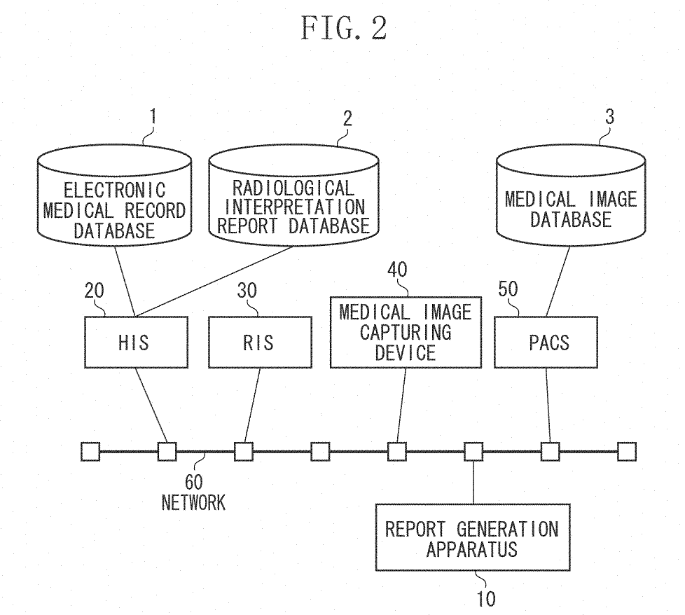

FIG. 2 is a block diagram illustrating an example of the configuration of a medical information system including the report generation apparatus 10 according to the present exemplary embodiment. The medical information system includes the report generation apparatus 10, a hospital information system (HIS) 20, a radiology information system (RIS) 30, a medical image capturing device 40, a picture archiving and communication system (PACS) 50, and a network 60.

In FIG. 2, the HIS 20, the RIS 30, the medical image capturing device 40, the PACS 50, and the report generation apparatus 10 communicate with each other to transmit and receive information via the network 60.

The HIS 20 is a system including a medical administration and billing system, a hospital appointment system, a medical information system, etc. The HIS 20 includes an electronic medical record database 1 and the radiological interpretation report database 2. For convenience, the radiological interpretation report database 2 is illustrated outside the HIS 20 in FIG. 2. The electronic medical record database 1 stores electronic medical records of patient medical information. The radiological interpretation report database 2 stores radiological interpretation reports generated by radiologists.

The RIS 30 is a system for radiology appointment, radiological interpretation management, inventory management, etc. in radiology departments. While the radiological interpretation report database 2 is managed by the HIS 20 in FIG. 2, there are cases in which the radiological interpretation report database 2 is managed by the RIS 30.

The medical image capturing device 40 includes one or more of a simple X-ray imaging apparatus (or roentgen apparatus), computed tomography (CT) apparatus, magnetic resonance imaging (MRI) apparatus, positron emission tomography (PET) apparatus, PET/CT apparatus, single photon emission computed tomography (SPECT) apparatus, ultrasonic image diagnosis apparatus, and optical coherence tomography (OCT) apparatus such as a fundus camera (or fundus imaging apparatus). The above-described apparatuses are installed in medical institutions, and the number of each apparatus is determined by each medical institution.

The PACS 50 is a system that electronically saves medical images captured by the medical image capturing device 40, performs a search, and communicates with other apparatuses. The PACS 50 includes a medical image database 3. For convenience, the medical image database 3 is illustrated outside the PACS 50 in FIG. 2.

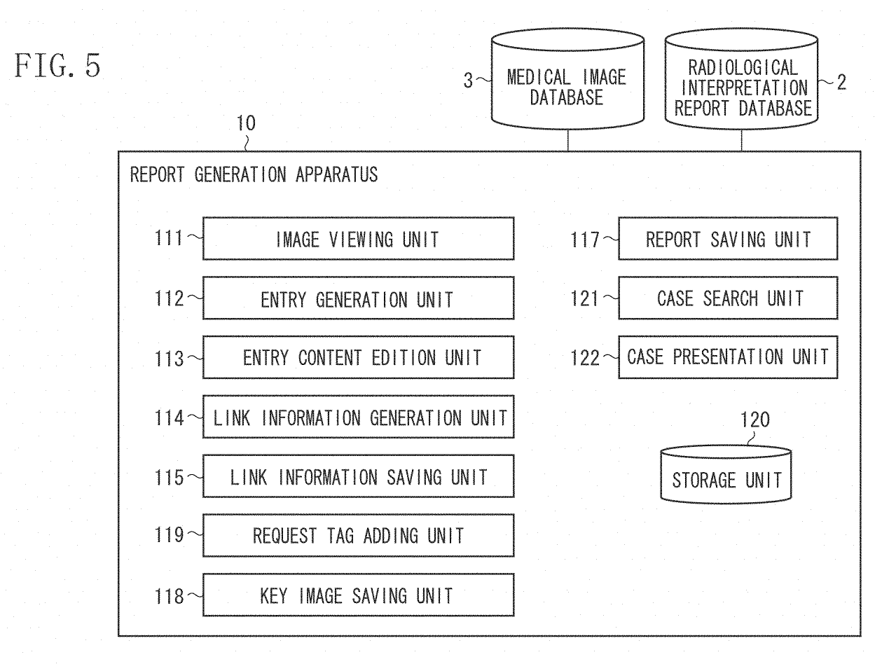

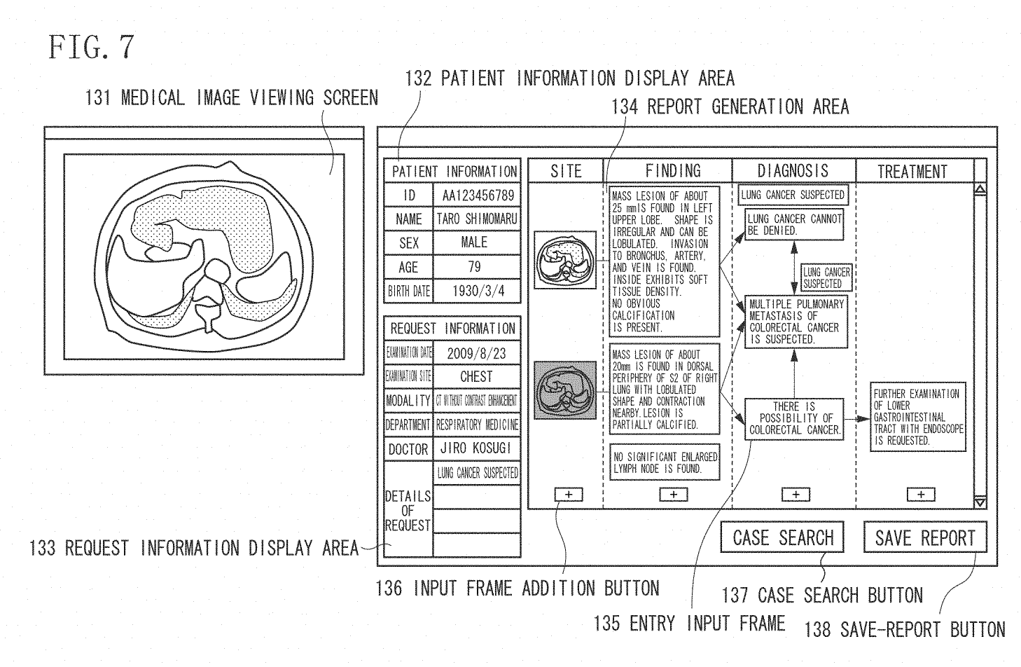

The report generation apparatus 10 generates, for example, a radiological interpretation report based on input from a radiologist. The report generation apparatus 10 includes various functions needed when a radiologist views a radiological interpretation target image to generate a radiological interpretation report. The functions of the report generation apparatus 10 will be described below with reference to FIGS. 5 and 7.

### (Network Architecture of Report)

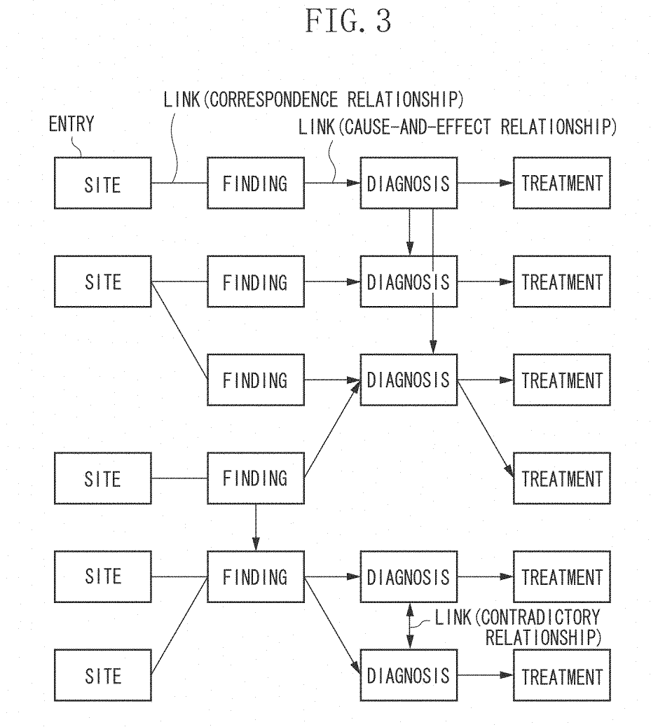

FIG. 3 illustrates an example of the concept of a report generated by the report generation apparatus 10.

A report generated by the report generation apparatus 10 according to the present exemplary embodiment includes, for example, a plurality of entries and link information about relationships between the entries.

For example, each entry (item) is classified into four categories of site, finding, diagnosis, and treatment based on the content. One or more of a sentence and an image by which a body site is identifiable is input to a site entry that is an entry indicating a site. Examples of an image input to the site entry include a soft copy of a radiological interpretation target image and address information for accessing a radiological interpretation target image. At the time of inputting an image to a site entry, image processing parameters and the like to be applied at the time of viewing the image can also be input to the site entry.

One or more sentences or words describing a lesion are input to a finding entry that is an entry indicating a finding.

One or more sentences or words describing a disease are input to a diagnosis entry that is an entry indicating a diagnosis.

One or more sentences or words describing a treatment are input to a treatment entry that is an entry indicating a treatment.

For example, the link information is classified into three categories of correspondence relationship, cause-and-effect relationship, and contradictory relationship based on the relationship between the entries. The correspondence relationship indicates that the two entries are interrelated. The cause-and-effect relationship indicates that one of the two entries is a cause and the other one is an effect. The contradictory relationship indicates that the two entries contradict each other. In the present embodiment, the link information is illustrated such that a line indicates a correspondence relationship, a single-headed arrow indicates a cause-and-effect relationship, and a double-headed arrow indicates a contradictory relationship.

A single entry can have a relationship with a plurality of other entries. Specifically, the relationships between the entries are many-to-many relationships.

### (Function Block)

FIG. 5 illustrates an example of a function block illustrating major functions of the report generation apparatus 10 according to the present exemplary embodiment.

The CPU 11 executes programs stored in the primary storage device 12 to function as an image viewing unit 111, an entry generation unit 112, an entry content edition unit 113, a link information generation unit 114, a link information saving unit 115, a report saving unit 117, a key image saving unit 118, a request tag addition unit 119, a case search unit 121, and a case presentation unit 122.

The image viewing unit 111 reads a radiological interpretation target image from the medical image database 3 via the PACS 50 and displays the read image on the output device 15. For example, the image viewing unit 111 displays the radiological interpretation target image on the output device 15 in response to a user instruction input via the input device 14. The image viewing unit 111 executes various processes related to image viewing, such as a change of a displayed image and application of image processing, in response to an operation performed by a radiologist via the input device 14.

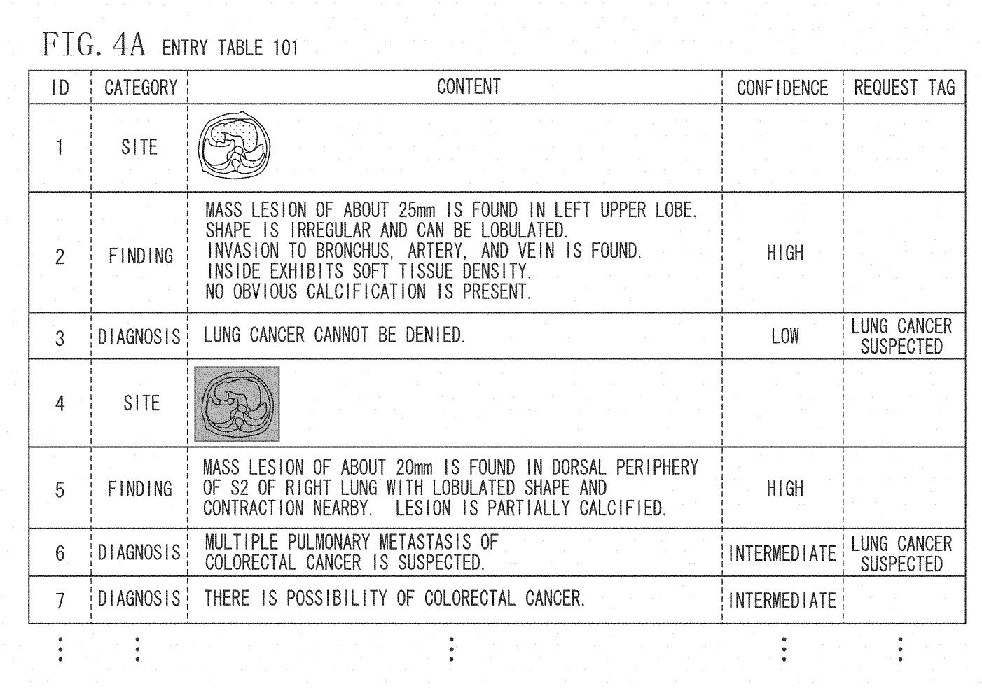

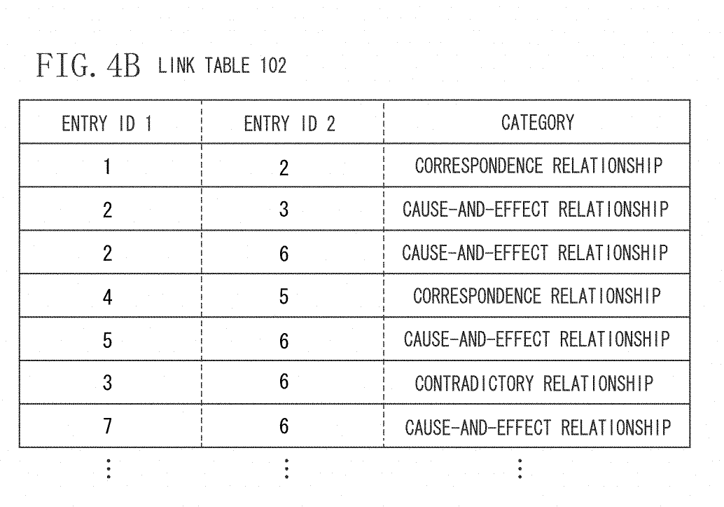

The entry generation unit 112 generates a new entry, automatically adds identification information (ID) and an item category to the generated entry, and saves the entry in an entry table 101 illustrated in FIG. 4A. The entry table 101 illustrated in FIG. 4A is saved in, for example, a storage unit 120. Alternatively, the entry generation unit 112 can generate a new entry in response to a user instruction input via the input device 14.

The entry content edition unit 113 edits the content of an entry and saves the edition result in a corresponding part of the entry table 101. At this time, if the category of the entry is the finding or diagnosis, the entry content edition unit 113 can also save the confidence of the content of the entry which indicates the likelihood of the content. For example, the entry content edition unit 113 estimates the confidence based on the word at the end of the sentence and automatically selects one of high, intermediate, low, and unknown. For example, the entry content edition unit 113 sets a lower confidence with respect to an entry with the term “cannot be denied” at the end of the sentence than the confidence of an entry with the term “suspected” at the end of the sentence.

In a case of an entry content written in a language other than Japanese, the entry content edition unit 113 can estimate the confidence based on a keyword other than the word at the end of the sentence. Alternatively, the confidence can be estimated using any other method or can be manually input. The confidence can be expressed using any method, such as a numerical value, that can indicate the likelihood of the content.

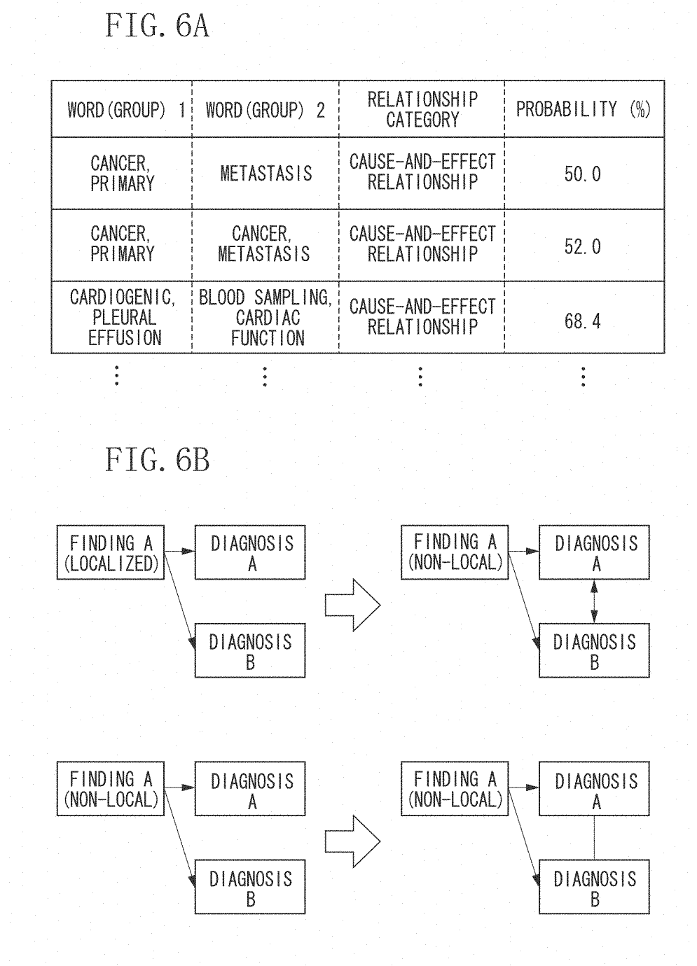

The link information generation unit 114 generates link information about a link between entries. The link information includes IDs of the two entries and the category of the link between the two entries. An item to which a link is to be added and the category of the link can be manually designated using a GUI illustrated in FIG. 7, which will be described below, or can be automatically determined by the link information generation unit 114. For example, the link information generation unit 114 can automatically add the cause-and-effect relationship as the category of a link between a diagnosis entry describing a disease that is a cause and a diagnosis entry describing a disease that is an effect using a cause-and-effect relationship table as illustrated in FIG. 6A. Based on the cause-and-effect relationship table illustrated in FIG. 6A, the link information generation unit 114 determines that the category of a link between two diagnosis entries is the cause-and-effect relationship if one of the two diagnosis entries includes the terms (words) “primary” and “cancer” and the other one includes the term “metastasis”. The link information generation unit 114 determines that the category of a link between two diagnosis entries is the cause-and-effect relationship if one of the two diagnosis entries includes the terms “primary” and “cancer” and the other one includes the terms “cancer” and “metastasis”. The link information generation unit 114 determines that the category of a link between two diagnosis entries is the cause-and-effect relationship if one of the two diagnosis entries includes the terms “cardiogenic” and “pleural effusion” and the other one includes the terms “blood sampling” and “cardiac function”. The link information generation unit 114 can determine the likelihood of a combination of terms included in a plurality of diagnosis entries as a probability based on the combination as illustrated in FIG. 6A. The examples illustrated in FIG. 6A are mere examples.

As, for example, illustrated in FIG. 6B, the link information generation unit 114 can automatically add link information between a plurality of diagnosis entries linked with a single finding entry. For example, if the content of the finding entry indicates a localized lesion, the link information generation unit 114 adds the contradictory relationship as the category of the link. If the content indicates a lesion other than a localized lesion, the link information generation unit 114 adds the correspondence relationship as the category of the link. As illustrated in FIG. 6B, for example, if a finding entry A indicates a localized lesion, the link information generation unit 114 adds the contradictory relationship as the category of a link between diagnosis entries A and B linked with the finding entry A. For example, the link information generation unit 114 analyzes sentences included in the finding entry A and determines whether the sentences include a term that indicates localization. For example, the terms that indicate localization are stored in advance in the storage unit 120.

As illustrated in FIG. 6B, if the finding entry A indicates a non-local lesion, the link information generation unit 114 adds the correspondence relationship as the category of the link between the diagnosis entries A and B linked with the finding entry A. For example, the link information generation unit 114 analyzes sentences included in the finding entry A and determines whether the sentences include a term that indicates non-localization. For example, the terms that indicate non-localization are stored in advance in the storage unit 120. The link information saving unit 115 saves in a link table 102 illustrated in FIG. 4B the link information generated by the link information generation unit 114. In the case in which the category of the link is the cause-and-effect relationship, the link information saving unit 115 saves the link information such that, for example, an entry ID 1 is the cause and an entry ID 2 is the result.

The report saving unit 117 saves the contents of the entry table 101 and the link table 102 as a radiological interpretation report in the radiological interpretation report database 2 via the HIS 20. The form of the radiological interpretation report can be any form that includes equivalent information to the entry table 101 and the link table 102. For example, the report saving unit 117 can convert the contents into a radiological interpretation report format defined by Extensible Markup Language (XML), etc. and save the converted contents. Specifically, the radiological interpretation report database 2 holds a mixture of radiological interpretation reports in which link information is added between diagnosis entries and radiological interpretation reports in which no link information is added between diagnosis entries.

The key image saving unit 118 saves an image viewed at the image viewing unit 111 as a key image in the report. In the present exemplary embodiment, for example, the key image saving unit 118 saves the key image as the content of the site entry generated by the entry generation unit 112.

The request tag addition unit 119 adds a word or sentence indicating details of a request as a request tag to a desired entry and saves the request tag in the entry table 101. A plurality of request tags can be added to a single entry. The details of a request are a message from the doctor having requested the radiologist to generate a radiological interpretation report and include patient complaints, symptoms, and the name of a diagnosis suspected by the doctor. In the example illustrated in FIG. 4A, the request tag “lung cancer suspected” is added to entry IDs 3 and ID 6 which are diagnosis entries.

The storage unit 120 temporarily stores the entry table 101 and the link table 102.

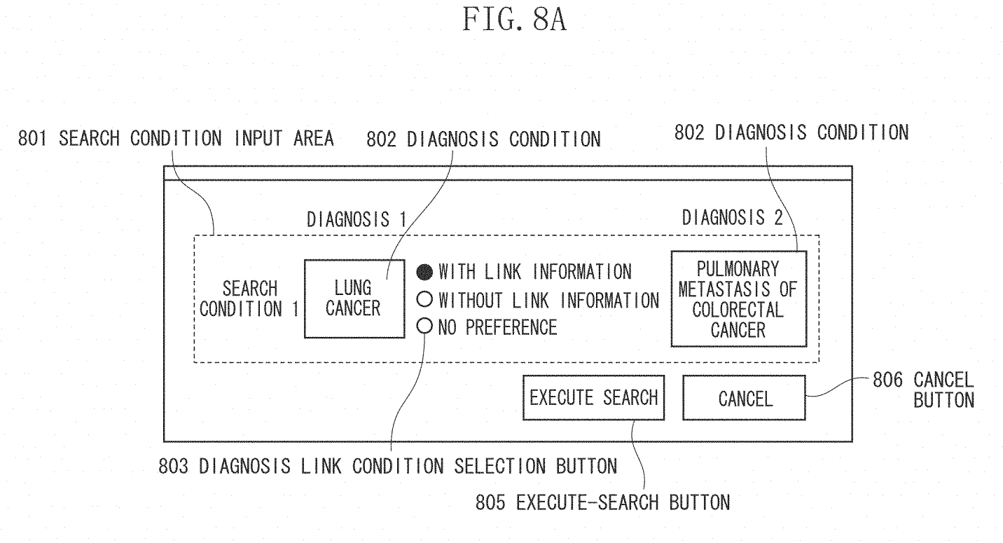

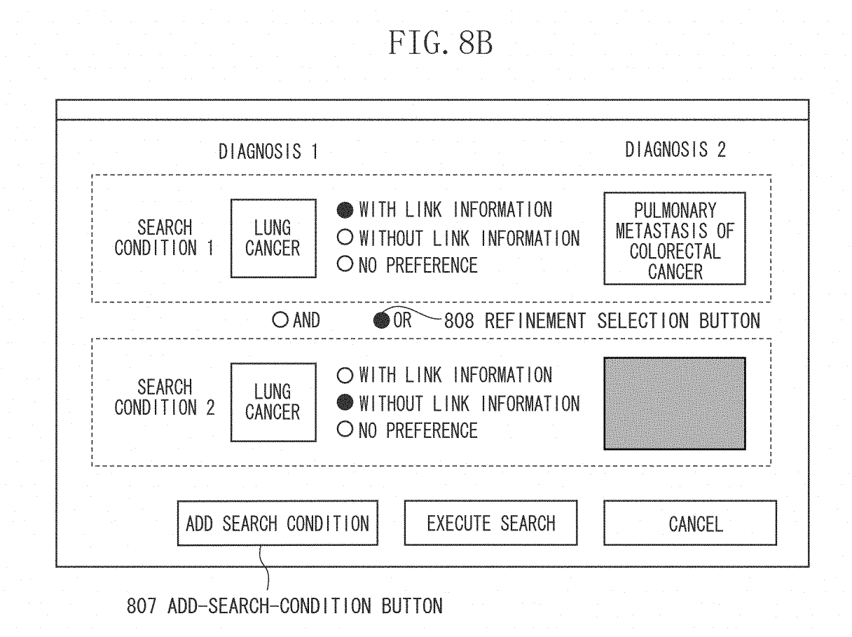

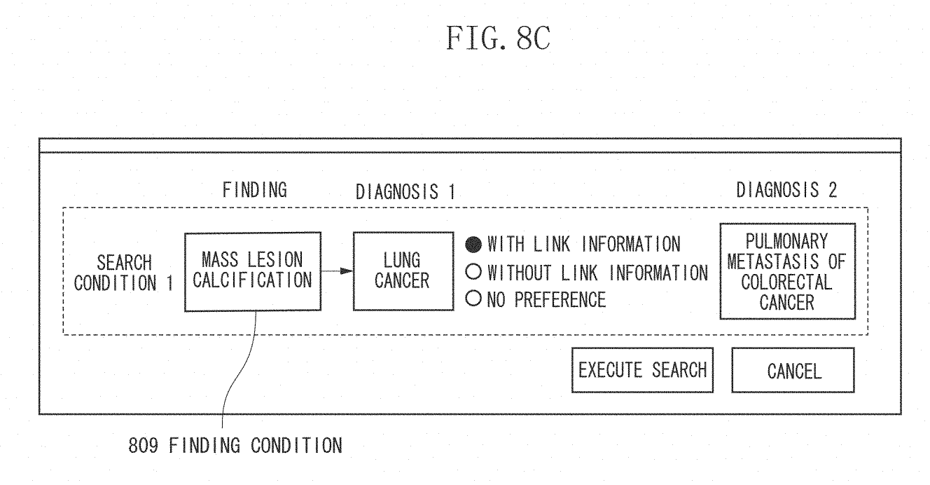

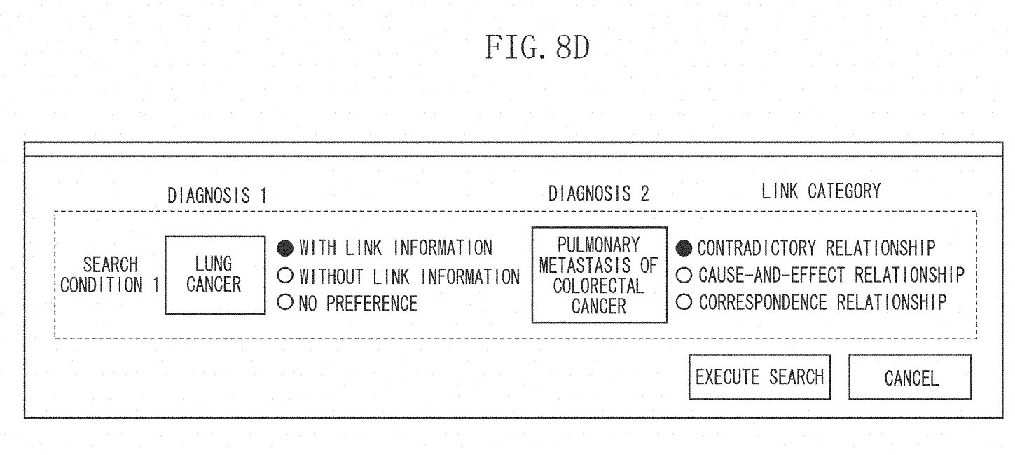

The case search unit 121 searches previous reports saved in the radiological interpretation report database 2 to search for a report corresponding to a search condition (search key) input to a search screen, which will be described below with reference to FIGS. 8A to 8D. For example, if diagnosis conditions 802 and 804 and “with link information” input by the user via a GUI illustrated in FIG. 8A are acquired, the case search unit 121 searches the radiological interpretation report database 2 to search for a case which includes diagnosis entries corresponding to the diagnosis conditions 802 and 804 and in which the diagnosis entries include link information. Specifically, the case search unit 121 corresponds to an example of an identification unit configured to identify, in a case where a first diagnosis item including a description about a diagnosis, a second diagnosis item including a description about a diagnosis which is different from the first diagnosis item, and link information indicating a relationship between the first diagnosis item and the second diagnosis item are acquired as a search key, a diagnosis report which includes the first diagnosis item and the second diagnosis item and in which the link information is added, from among a plurality of diagnosis reports stored in a storage unit.

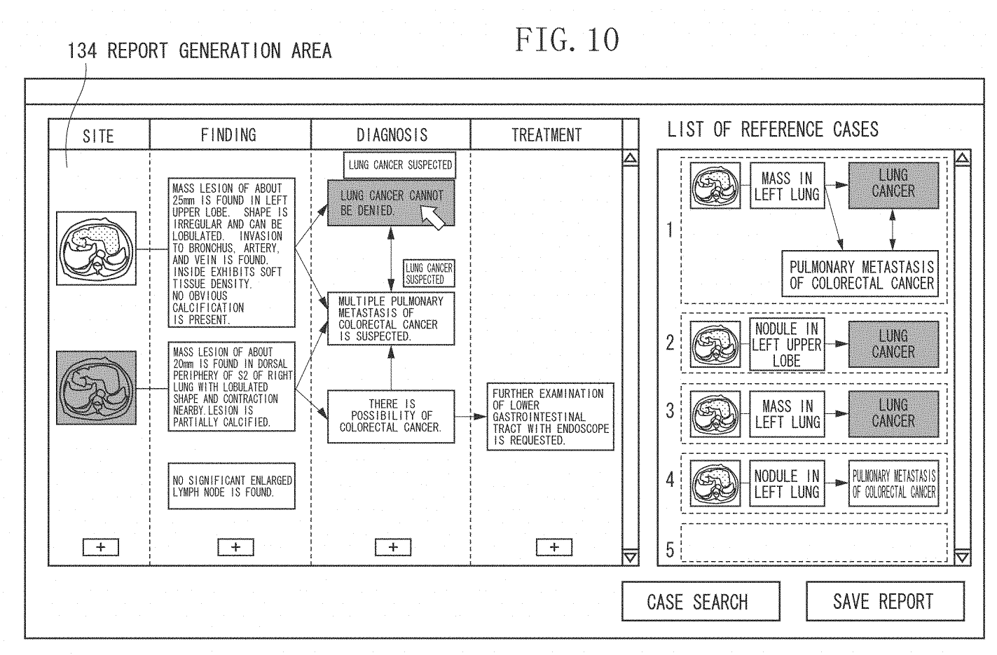

The case presentation unit 122 displays as a case the searched report acquired by the case search unit 121 on the output device 15, for example, as illustrated in FIG. 10. Specifically, the report generation apparatus 10 including the case presentation unit 122 corresponds to an example of an information processing apparatus configured to output a specific report from among a plurality of diagnosis reports. Further, the case presentation unit 122 and/or the output device 15 corresponds to an output unit configured to output a diagnosis report identified by the identification unit.

The case presentation unit 122, for example, displays on the output device 15 each entry according to the entry table 101 included in the searched report and displays on the output device 15 the link information about the links between the entries according to the contents of the link table 102 included in the searched report. The case presentation unit 122 can determine whether to display the link information on the output device 15 based on the category of the link information. For example, the case presentation unit 122 can display on the output device 15 no link information added between diagnosis entries. Further, for example, the case presentation unit 122 can display manually generated link information while displaying no automatically generated link information on the output device 15.

### (Example of User Interface)

FIG. 7 illustrates an example of a GUI of the report generation apparatus 10 according to the present exemplary embodiment.

The output device 15 displays a medical image viewing screen 131 including a radiological interpretation target image. Further, the image viewing unit 111 acquires the radiological interpretation target image and displays the acquired radiological interpretation target image on the output device 15 in response to a user operation performed on the medical image viewing screen 131.

A patient information display area 132 displays information about the patient who is the subject of the radiological interpretation target image. Examples of the patient information include a patient ID, name, age, and previous illness history.

A request information display area 133 displays information about the requesting department, requested examination, and details of the request from the doctor. In the present exemplary embodiment, the details of the request are divided in advance into separate items, and each item is selectable as a request tag. The details of the request that are expressed by sentences containing a mixture of a plurality of topics can be divided manually or automatically into separate items and displayed in the request information display area 133.

A report generation area 134 is divided into site, finding, diagnosis, and treatment areas, and an entry input frame 135 for inputting the content of the corresponding entry and an input frame addition button 136 for adding another entry input frame 135 are displayed in each of the site, finding, diagnosis, and treatment areas.

The entry input frame 135 is associated with a single entry and receives an operation to edit the content of the associated entry. The function of the entry content edition unit 113 is executed through an operation performed on the entry input frame 135.

At the press of the input frame addition button 136, the function of the entry generation unit 112 is executed, and the entry generation unit 112 generates a new entry. At the same time, the entry generation unit 112 displays on the output device 15 the entry input frame 135 corresponding to the generated entry. In the present exemplary embodiment, the input frame addition button 136 is prepared for each category of the item, and the entry generation unit 112 generates an entry of a category corresponding to a pressed button.

Alternatively, the addition of the entry input frame 135 can be conducted by a key operation via the input device 14. For example, the entry generation unit 112 can generate a diagnosis entry at the press of a TAB key while the content of the finding entry is edited. The entry generation unit 112 can generate another finding entry at the double-press of a RETURN key while the content of the finding entry is edited.

The entry input frame 135 is selected via the input device 14. A plurality of entry input frames 135 can be simultaneously selected. Further, if the selected entry input frame 135 is dragged and dropped onto another entry input frame 135, the report generation apparatus 10 determines that an instruction to add link information about a link between an entry corresponding to the dragged entry input frame 135 and an entry corresponding to the entry input frame 135 onto which the dragged entry input frame 135 is dropped is given. A different method can be used to add link information between entries. Further, the category of link information can be added by the user either after or before the execution of dragging and dropping. The category of link information can be input via a window on which a category is selectable from a pull-down menu, or various publicly-known methods can be used to input the category of link information. If an instruction to add link information or the like is given, the link information generation unit 114 and the link information saving unit 115 sequentially execute the above-described processing.

If an image is dragged and dropped from the medical image viewing screen 131 to the report generation area 134, the function of the key image saving unit 118 is executed. Then, the entry generation unit 112 generates a site entry and displays the corresponding entry input frame 135 on the output device 15. Further, the entry generation unit 112 simultaneously generates a finding entry having the correspondence relationship with the generated site entry. Then, the entry generation unit 112 displays the entry input frame 135 corresponding to the finding entry on the output device 15. Further, if an image is dragged and dropped from the medical image viewing screen 131 to the existing entry input frame 135, the key image is added to the content of the entry onto which the image is dropped.

If the request tag is dragged and dropped from the request information display area 133 to the entry input frame 135, the request tag addition unit 119 adds the dragged request tag to the entry onto which the request tag is dropped. Further, in the present exemplary embodiment, the output device 15 displays the request tag on each entry input frame 135 so that the user can recognize the content of the request tag added to each entry. The request tag does not always have to be displayed on the entry input frame 135.

At the press of a case search button 137, the output device 15 displays a window of a search screen illustrated in FIGS. 8A to 8D. If user input is received via the search screens illustrated in FIGS. 8A to 8D, the processing of the case search unit 121 is executed. Further, the processing of the case presentation unit 122 is executed based on a result of a search performed by the case search unit 121.

At the press of a save-report button 138, the function of the report saving unit 117 is executed.

### (Case Search)

FIG. 8A illustrates an example of the GUI via which a search condition for the execution of the case search unit 121 is input.

A search condition input area 801 receives input of a search condition (search key). Specifically, the case search unit 121 receives a search condition via the search condition input area 801. The search condition input area 801 includes the diagnosis condition 802 and 804 and a diagnosis link condition selection button 803.

The respective contents of the diagnosis entries are input to the diagnosis condition 802 and 804. The diagnosis link condition selection button 803 is a button for selecting a condition of a link between the diagnosis entries specified in the diagnosis conditions 802 and 804. The condition of the link includes, for example, three options which are “with link information”, “without link information”, and “no preference”. The options are not limited to the above-described options and can be, for example, two options which are “with link information” and “without link information”. Alternatively, only the option “with link information” is made available, and if the option is selected by the user, the case search unit 121 determines that “with link information” is selected, whereas if the option is not selected by the user, the case search unit 121 determines that “without link information” is selected. Alternatively, the case search unit 121 can control the output device 15 such that while the content is allowed to be input to the diagnosis condition 804 in the case where “with link information” is selected, the content is not allowed to be input in the other case.

The case search unit 121 can control the output device 15 such that the diagnosis link condition selection button 803 is selectable only if the content is input to the diagnosis condition 802. Alternatively, the case search unit 121 can control the output device 15 such that the diagnosis link condition selection button 803 is made non-selectable only after “no preference” is selected in the case where no content is input to the diagnosis condition 802.

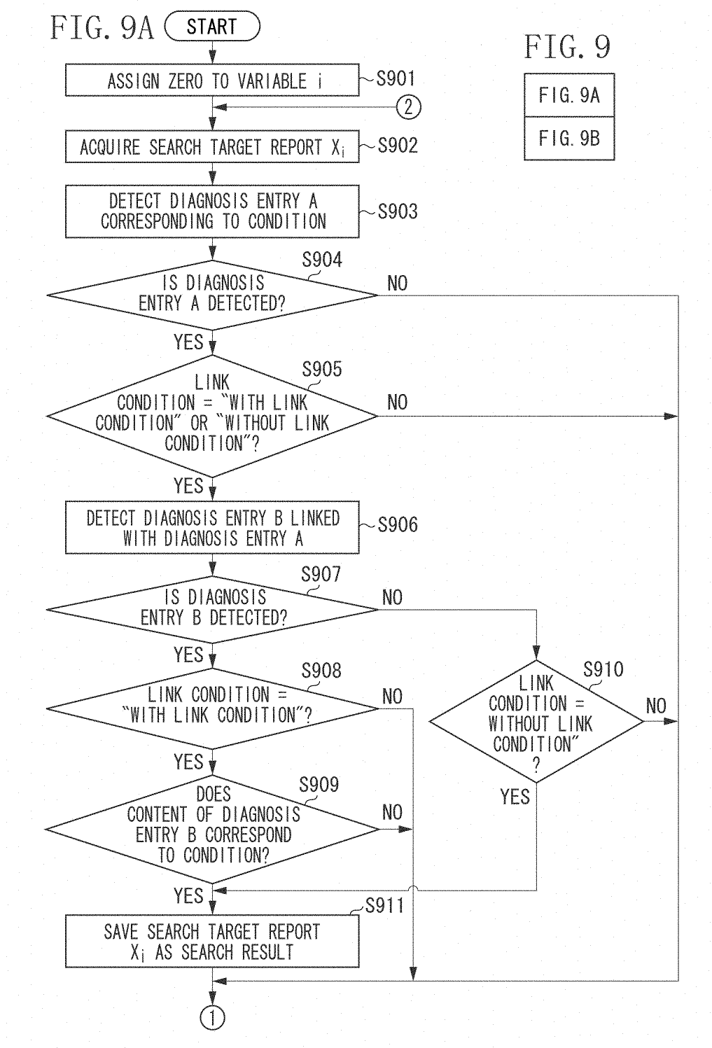

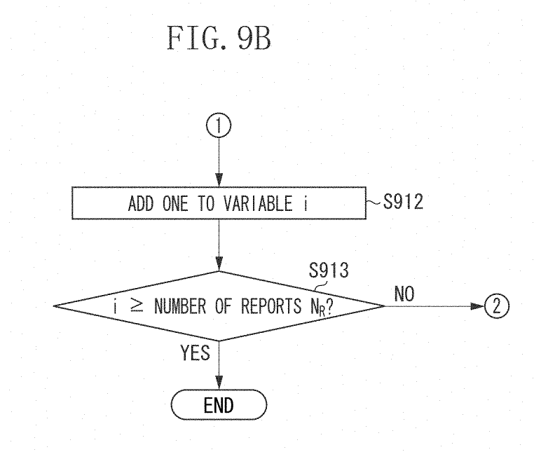

At the press of an execute-search button 805, the case search unit 121 executes a case search using, for example, a method described below with reference to FIGS. 9A and 9B.

At the press of a cancel button 806, the output device 15 closes the search screen illustrated in FIGS. 8A to 8D.

FIGS. 9A and 9B are a flowchart illustrating an example of a process of executing a case search based on the search conditions input via the GUI illustrated in FIG. 8A.

In the present exemplary embodiment, a target of the case search is, for example, the reports saved in the radiological interpretation report database 2. Hereinafter, the number of reports saved in the radiological interpretation report database 2 will be denoted by NR, and the n-th report will be denoted by Xₙ.

In step S901, the case search unit 121 assigns zero to a variable i.

In step S902, the case search unit 121 acquires a search target report Xᵢ from the radiological interpretation report database 2. For example, the case search unit 121 acquires the entry table 101 and the link table 102 from the search target report Xᵢ.

In step S903, the case search unit 121 searches the search target report Xᵢ to detect the diagnosis entry A corresponding to the content of the diagnosis condition 802. Specifically, the case search unit 121 refers to the entry table 101 to detect the diagnosis entry A corresponding to the content of the diagnosis condition 802. The case search unit 121 determines whether the content corresponds based on whether the word expressing the disease name is the same. Alternatively, the case search unit 121 can determine that the diagnosis entry A corresponds to the content of the diagnosis condition 802 in the case where the diagnosis entry A includes a word or sentence indicating the same diagnosis content even if the word is not exactly the same. Alternatively, any other method can be used to determine whether the content corresponds.

In step S904, the case search unit 121 determines whether the diagnosis entry A is detected from the search target report Xᵢ in step S903. If the diagnosis entry A is detected (YES in step S904), the processing proceeds to step S905. On the other hand, if no diagnosis entry A is detected (NO in step S904), the processing proceeds to step S912.

In step S905, the case search unit 121 determines whether “with link information” or “without link information” is selected in the diagnosis link condition selection button 803. If “with link information” or “without link information” is selected (YES in step S905), the processing proceeds to step S906. On the other hand, neither “with link information” nor “without link information” is selected (NO in step S905), the processing proceeds to step S912.

In step S906, the case search unit 121 searches the content of the search target report Xᵢ to detect another diagnosis entry B linked with the diagnosis entry A detected in step S903. For example, the case search unit 121 refers to the content of the link table 102 acquired from the radiological interpretation report database 2 to detect the diagnosis entry B linked with the diagnosis entry A.

In step S907, the case search unit 121 determines whether the diagnosis entry B is detected in step S906. If the diagnosis entry B is detected (YES in step S907), the processing proceeds to step S908. On the other hand, if no diagnosis entry B is detected (NO in step S907), the processing proceeds to step S910.

In step S908, the case search unit 121 determines whether “with link information” is selected in the diagnosis link condition selection button 803. If “with link information” is selected (YES in step S908), the processing proceeds to step S909. On the other hand, if “with link information” is not selected (NO in step S908), the processing proceeds to step S912.

In step S909, the case search unit 121 determines whether the content of the diagnosis entry B detected in step S906 corresponds to the content of the diagnosis condition 804. The determination of whether the contents correspond is performed using, for example, the same method as the method of determining whether the diagnosis entry A corresponds to the diagnosis condition 802. In the case where no content is described in the diagnosis condition 804, the case search unit 121 can determine that the content of the diagnosis condition 804 corresponds to the content of the diagnosis entry B regardless of the content of the diagnosis entry B. If the case search unit 121 determines that the contents correspond (YES in step S909), the processing proceeds to step S911. On the other hand, if the case search unit 121 determines that the contents do not correspond (NO in step S909), the processing proceeds to step S912.

In step S910, the case search unit 121 determines whether “without link information” is selected in the diagnosis link condition selection button 803. If “without link information” is selected (YES in step S910), the processing proceeds to step S911. On the other hand, if “without link information” is not selected (NO in step S910), the processing proceeds to step S912.

In step S911, the case search unit 121 temporarily saves the search target report Xᵢ as a search result, in, for example, the storage unit 120. The case search unit 121 can temporarily save the entire search target report Xᵢ in the storage unit 120 or can extract a part of the search target report Xᵢ that includes the diagnosis conditions 802 and 804 and save the extracted part in the storage unit 120.

In step S912, the case search unit 121 adds one to the variable i.

In step S913, the case search unit 121 compares the variable i with the number of reports NR saved in the radiological interpretation report database 2. If the variable i is not less than the number of reports N_{R} (YES in step S913), the case search process is ended. On the other hand, if the variable i is less than the number of reports N_{R} (NO in step S913), the processing proceeds to step S902.

While the foregoing describes that the case search unit 121 sequentially searches the reports included in the radiological interpretation report database 2, the search can be parallelized, etc. to enable high-speed processing. Further, the case search unit 121 can refine the reports based on the captured site, image capturing method, etc. and then execute search processing.

Further, in the case where a plurality of diagnosis entries A is detected in step S903, steps S905 to S909 are executed on each of the diagnosis entries A. As a result, if any one of the diagnosis entries A corresponds to the condition, the case search unit 121 employs the search target report Xᵢ as a search result.

Further, in the case where a plurality of diagnosis entries B is detected in step S906, steps S908 to step S909 are executed on each of the diagnosis entries B. As a result, if any one of the diagnosis entries B corresponds to the condition, the case search unit 121 employs the search target report Xᵢ as a search result.

After executing the process illustrated in FIGS. 9A and 9B, the case presentation unit 122 outputs the search result stored in the storage unit 120 to the output device 15 and displays the search result on the output device 15. The case presentation unit 122 displays the report saved temporarily in step 911 in FIG. 9A as a reference case list on the output device 15 as a case search result, for example as illustrated in FIG. 10. As indicated by the reference case list in FIG. 10, the output diagnosis report includes at least one of a first finding item corresponding to a first diagnosis item, a second finding item corresponding to a second diagnosis item, a first image corresponding to the first finding item, and a second image corresponding to the second finding item.

The list displays, for example, the part of the content of each case that is designated as the search condition. The displayed contents of the list can be representative images of the respective cases or a list of diagnoses. As illustrated in FIG. 8A, since the diagnosis conditions 802 and 804 are respectively “lung cancer” and “pulmonary metastasis of colorectal cancer” and the diagnosis link condition is “with link information”, the case in which link information indicating the contradictory relationship is added between “lung cancer” and “colorectal cancer, pulmonary metastasis” is displayed at the top of the reference case list as the case search result in FIG. 10.

The order of display on the list can be determined according to any condition. For example, the case presentation unit 122 can sort the cases according to the diagnosis confidence or according to the number of findings linked with the diagnosis.

The above-described process is performed so that only the cases in which a link is added between a specific diagnosis entry and another diagnosis entry are acquired in a case search. For example, when a doctor vacillates between “lung cancer” and “pulmonary metastasis of colorectal cancer”, which are possible diagnoses from a finding and have a contradictory relationship, while generating a radiological interpretation report, the present exemplary embodiment enables a search for reference cases in which an explicit relationship is added between “lung cancer” and “pulmonary metastasis of colorectal cancer”. For example, when a case in which link information indicating the contradictory relationship is added between “lung cancer” and “pulmonary metastasis of colorectal cancer” is found as a result of a search, the found case helps the doctor in making a decision that lung cancer and colorectal cancer cannot be identified uniquely from the finding.

Specifically, the present exemplary embodiment enables a search for suitable cases desired by doctors. This reduces the burden of searching for desired cases from search results on the doctors.

Further, the present exemplary embodiment enables acquisition of only the cases in which there is no diagnosis entry linked with a specific diagnosis entry. For example, when a doctor vacillates between “lung cancer” and “pulmonary metastasis of colorectal cancer”, which are possible diagnoses from a finding and have a contradictory relationship, while generating a radiological interpretation report, a case in which the diagnosis entry “lung cancer” has no link information helps the doctor in confirming “lung cancer”. The second and third cases from the top on the reference case list in FIG. 10 are examples of the case that helps to confirm “lung cancer”. Further, a case in which the diagnosis entry “pulmonary metastasis of colorectal cancer” has no link information helps to confirm “pulmonary metastasis”. The fourth case from the top on the reference case list in FIG. 10 is an example of the case that helps to confirm “pulmonary metastasis”.

Specifically, the present exemplary embodiment enables a search for suitable cases desired by doctors. This reduces the burden of searching for desired cases from search results on the doctors.

In the present exemplary embodiment, the content of a single diagnosis entry and link information about a link between the diagnosis entry and another diagnosis entry are determined as a single search condition, and a case search is performed based on the single search condition. Alternatively, a plurality of search conditions can be set to enable a complex search. FIG. 8B illustrates an example of a GUI for inputting a plurality of search conditions in the foregoing case. The output device 15 displays an additional search condition at the press of a search condition addition button 807. Whether to use the plurality of search conditions in refinement is selected using a refinement selection button 808.

At the press of the execute-search button 805, the case search unit 121 executes a search for a case corresponding to the respective search conditions according to the method described above with reference to FIGS. 9A and 9B. The case search unit 121 performs a logical AND or OR operation on the search results to obtain a final search result.

Further, while only the content of a diagnosis entry is input as a search condition in the present exemplary embodiment, the content of a finding entry linked with the diagnosis entry can also be input. FIG. 8C illustrates an example of a GUI for inputting search conditions in the foregoing case. If the content of a finding condition 809 is input, the case search unit 121 determines whether any one of the finding entries linked with the diagnosis entry A detected in step S903 in FIG. 9A corresponds to the finding condition 809. The case search unit 121 leaves the diagnosis entry A as a search result if any one of the finding entries corresponds to the finding condition 809, whereas the case search unit 121 excludes the diagnosis entry A from the search result if none of the finding entries corresponds to the finding condition 809.

In a case where a value is input to the finding condition 809 and no value is input to the diagnosis conditions 802 and 804 on the GUI illustrated in FIG. 8C, the case search unit 121 can search only for cases that correspond to the finding condition 809. Specifically, a search for examples of diagnoses corresponding to the finding can be performed.

Similarly, image information can also be input. In this case, the case search unit 121 refines the search result using feature amounts of the image, etc.

Further, the GUIs illustrated in FIGS. 8B and 8C can be combined. Specifically, a finding condition can be input with respect to each of a plurality of search conditions.

While the case search unit 121 only determines the presence/absence of link information as a search condition in the present exemplary embodiment, the link category can be added to the search condition. FIG. 8D illustrates an example of a GUI for use in the foregoing case. For example, the case search unit 121 can search for cases in which the link information between the diagnosis conditions 802 and 804 is one of the contradictory relationship, cause-and-effect relationship, and correspondence relationship. Specifically, the case search unit 121 is to search for cases having link information of the same category as the category of the link information designated on the GUI illustrated in FIG. 8D. Specifically, in a case where information indicating a category of the link information is included in the search key, the case search unit 121 identifies a diagnosis report which includes the first diagnosis item and the second diagnosis item and includes the link information of the same category as the category included in the search key.

Specifically, searches for cases desired by doctors are performed more suitably. While the three categories are described as examples of the category of the link information, two or less or four or more categories can be used. Further, either a single category or a plurality of categories of link information can be selected as a search condition. Further, the GUI illustrated in FIG. 8D can be combined with the GUI illustrated in FIG. 8B and/or the GUI illustrated in FIG. 8C.

Further, the options that the diagnosis is “not confirmed” and that the diagnosis is “confirmed” can be prepared with the diagnosis link condition selection button 803 in place of or in addition to the options “with link information” and “without link information”. At this time, information about whether the diagnosis is confirmed can be set separately to the diagnosis condition, and the case search unit 121 can perform a case search using the set content. The radiological interpretation reports saved in the radiological interpretation report database 2 contain information about whether the diagnosis is confirmed. Further, the case search unit 121 can link the diagnosis entry with information of another system such as an electronic medical record to determine whether the diagnosis is confirmed based on the linked information.

Further, the diagnosis conditions 802 and 804 can still be input even if “without link information” is selected in the diagnosis link condition selection button 803. In this case, the case search unit 121 can detect the diagnosis entry B which is not linked with the diagnosis entry A in step S906 in FIG. 9A. Then, even if the case search unit 121 determines that the without-link-condition option is selected in step S908, the processing still proceeds to step S909. The above-described process enables a search for a case that includes the diagnosis conditions 802 and 804 and has no link information between the diagnosis conditions 802 and 804. Specifically, in a case where the search key does not include link information, the case search unit 121 identifies a diagnosis report that includes the first and second diagnosis items and does not include the link information.

In a case in which the diagnosis link condition selection button 803 does not include the option “no preference”, if “without link information” is selected in the diagnosis link condition selection button 803, the case search unit 121 can detect the diagnosis entry B which is not linked with the diagnosis entry A and the diagnosis entry B which is linked with the diagnosis entry A in step S906 in FIG. 9A. Then, regardless of whether the case search unit 121 determines that the without-link-condition option is selected or that the with-link-condition option is selected in step S908, the processing proceeds to step S909. The above-described process enables a search for a case that includes the diagnosis conditions 802 and 804 and has no link information between the diagnosis conditions 802 and 804 and a case that includes the diagnosis conditions 802 and 804 and has link information between the diagnosis conditions 802 and 804. Specifically, in a case where the search key does not include the link information, the case search unit 121 identifies a diagnosis report that includes the first and second diagnosis items and does not include the link information and a diagnosis report that includes the first and second diagnosis items and includes the link information.

In a second exemplary embodiment, a method of realizing a report system capable of automatically searching for a necessary case based on the report generation status will be described.

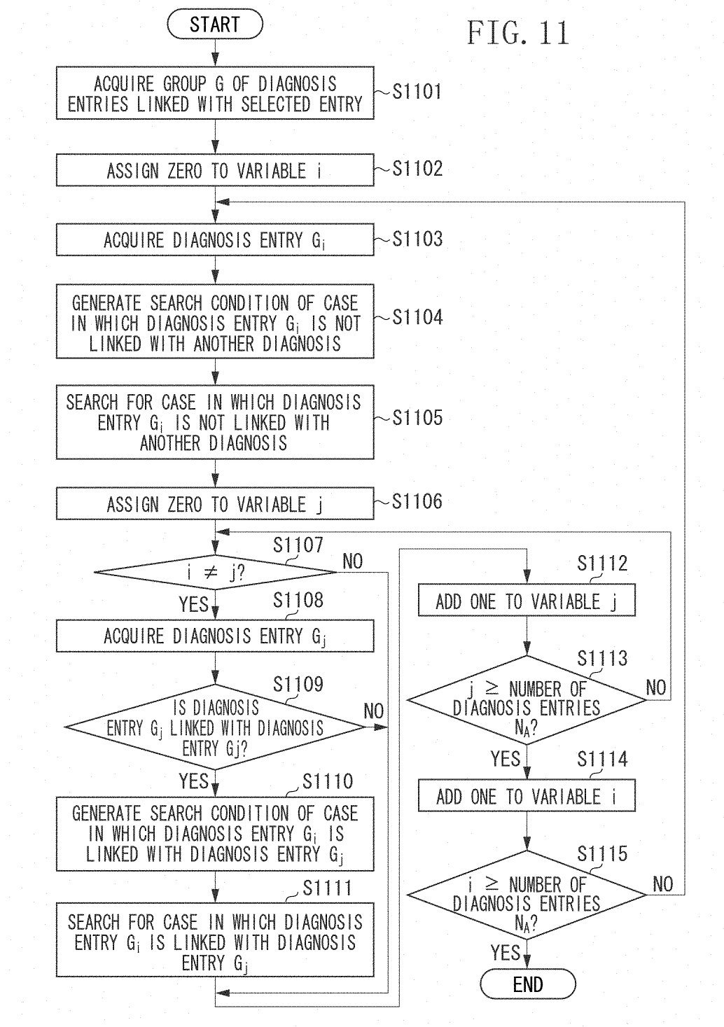

In the second exemplary embodiment, the case search unit 121 automatically searches for a case using a method described below with reference to FIG. 11 without the use of the search windows illustrated in FIGS. 8A to 8D.

In the second exemplary embodiment, the timing of execution of the case search unit 121 is, for example, the timing of the press of the case button 137 illustrated in FIG. 7 as in the first exemplary embodiment. The timing of execution of a case search by the case search unit 121 can be automatically determined. For example, the case presentation unit 122 can constantly display a case search result on the output device 15 as illustrated in FIG. 10 and automatically search for a case and present the case when the user selects an input frame of the entries.

FIG. 11 is a flowchart illustrating an example of a process of automatically searching for a case in the case search unit 121 according to the second exemplary embodiment. The following description includes a description of the process in which the case search unit 121 automatically generates a search condition based on the report generation status, and the search condition to be generated will be described with reference to the GUIs illustrated in FIGS. 8A to 8D for convenience. It should be noted, however, that the GUIs illustrated in FIGS. 8A to 8D are not displayed visibly to the user and only equivalent data is held in the background.

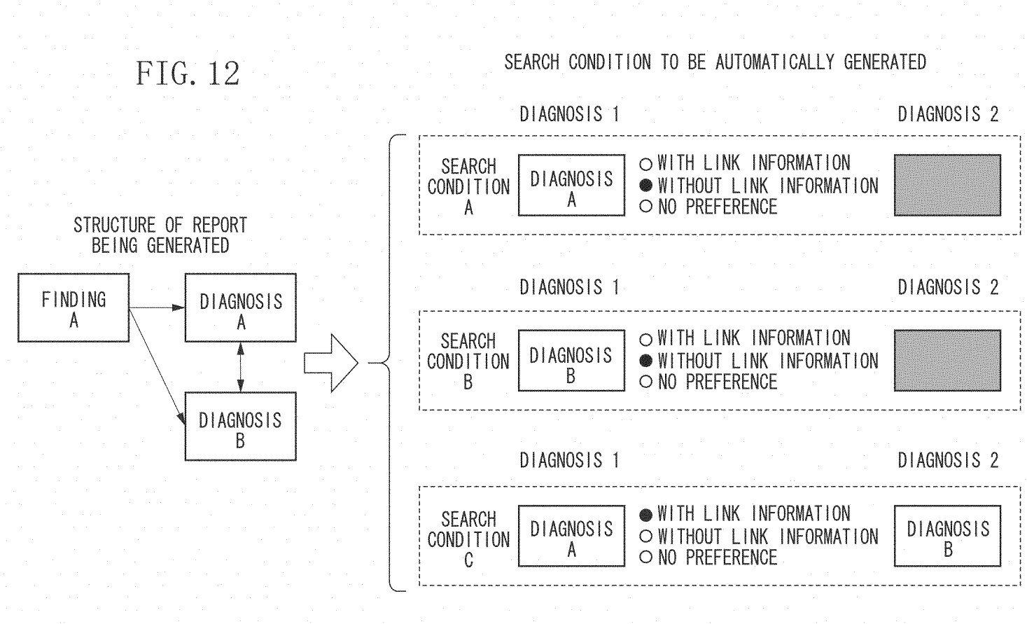

In step S1101, the case search unit 121 acquires a group G of diagnosis entries linked directly or indirectly with the entry selected in the report generation area 134. Hereinafter, the n-th diagnosis entry in the group G will be denoted by Gₙ. Further, the number of diagnosis entries included in the group G will be denoted by NA. For example, if the entry of the finding A is selected in the report architecture being generated in FIG. 12, the entries of the diagnoses A and B are acquired as the group G of diagnosis entries.

In step S1102, the case search unit 121 assigns zero to the variable i.

In step S1103, the case search unit 121 acquires a diagnosis entry Gᵢ.

In step S1104, the case search unit 121 generates a search condition for searching for a case in which the diagnosis entry Gᵢ is not linked with another diagnosis. Specifically, the case search unit 121 inputs the content of the diagnosis entry Gᵢ to the diagnosis condition 802, and the diagnosis link condition selection button 803 selects “without link information”. Specifically, in step S1104, the case search unit 121 can generate the search condition A or B illustrated in FIG. 12.

In a case in which there is a finding entry linked with the diagnosis entry Gᵢ, the case search unit 121 can add the content of the finding entry to the search condition as in the finding condition 809 illustrated in FIG. 8C.

In step S1105, the case search unit 121 executes the search processing described above with reference to FIGS. 9A and 9B based on the search condition generated in step S1104.

In step S1106, the case search unit 121 assigns zero to a variable j.

In step S1107, the case search unit 121 compares the values of the variables i and j. If i≠j is satisfied (YES in step S1107), the processing proceeds to step S1108. On the other hand, if i≠j is not satisfied (NO in step S1107), the processing proceeds to step S1112.

In step S1108, the case search unit 121 acquires a diagnosis entry Gⱼ.

In step S1109, the case search unit 121 determines whether the diagnosis entry Gᵢ is linked with the diagnosis entry Gⱼ. For example, the case search unit 121 refers to the link table 102 to determine whether there is link information between the diagnosis entries Gᵢ and Gⱼ. In the example illustrated in FIG. 12, the case search unit 121 determines whether there is link information between the entries of the diagnoses A and B.

If the diagnosis entry Gᵢ is linked with the diagnosis entry Gⱼ (YES in step S1109), the processing proceeds to step S1110. On the other hand, if the diagnosis entry Gᵢ is not linked with the diagnosis entry Gⱼ (NO in step S1109), the processing proceeds to step S1112. In the example illustrated in FIG. 12, link information indicating the contradictory relationship exists between the entries of the diagnoses A and B, so the processing proceeds to step S1110.

In step S1110, the case search unit 121 generates a search condition for searching for a case in which the diagnosis entry Gᵢ is linked with the diagnosis entry Gⱼ. Specifically, the case search unit 121 inputs the content of the diagnosis entry Gᵢ to the diagnosis condition 802, and the diagnosis link condition selection button 803 selects “with link information”. Further, the case search unit 121 inputs the content of the diagnosis entry Gⱼ to the diagnosis condition 804. Specifically, the case search unit 121 generates a search condition C illustrated in FIG. 12 in step S1110.

In a case in which there is a finding entry linked with the diagnosis entry Gᵢ, the case search unit 121 can add the content of the finding entry to the search condition as in the finding condition 809 illustrated in FIG. 8C.

In step S1111, the case search unit 121 executes the search processing described above with reference to FIGS. 9A and 9B based on the search condition generated in step S1110.

In step S1112, the case search unit 121 adds one to the variable j.

In step S1113, the case search unit 121 compares the variable j with the number of diagnosis entries NA included in the group G. If j≥N_{A} is satisfied (YES in step S1113), the processing proceeds to step S1114. On the other hand, if j≥N_{A} is not satisfied (NO in step S1113), the processing proceeds to step S1107.

In step S1114, the case search unit 121 adds one to the variable i.

In step S1115, the case search unit 121 compares the variable i with the number of diagnosis entries N_{A} included in the group G. If i≥N_{A} is satisfied (YES in step S1115), the process is ended. On the other hand, if i≥N_{A} is not satisfied (NO in step S1115), the processing proceeds to step S1103.

In the above description, a link between each diagnosis entry included in the group G and every one of the other diagnosis entries included in the group G is determined to search for corresponding cases. Alternatively, a group of previously-searched diagnosis entries can be stored to avoid a duplicated search so that the processing is shortened. Further, only the entries that are directly linked with the selected diagnosis entry can be set as a search target.

After the process described above with reference to FIG. 11 is executed, the case presentation unit 122 displays a search result list on the output device 15 as illustrated in FIG. 10.

The case presentation unit 122 can highlight the part of the contents of the listed cases that corresponds to the entry selected in the report generation area 134.

The display order of the listed search results is, for example, the order in which the search results are acquired in the process illustrated in FIG. 11. It is also certainly possible to sort the search results according to any other condition. For example, the network architecture mainly formed by the entry selected in the report generation area 134 is compared with the network architecture of the cases of the search results to sort the search results in descending order according to correspondence.

The above-described exemplary embodiment produces an advantage similar to that produced by the first exemplary embodiment and automatically presents useful cases according to the radiological interpretation report generation status.

Specifically, when there is a plurality of possible diagnoses in radiological interpretation, a case as a reference for confirming a diagnosis and a case as a reference for ending a diagnosis without confirming the diagnosis are simultaneously searched and presented in the present exemplary embodiment. This enables automatic presentation of useful cases according to the radiological interpretation report generation status.

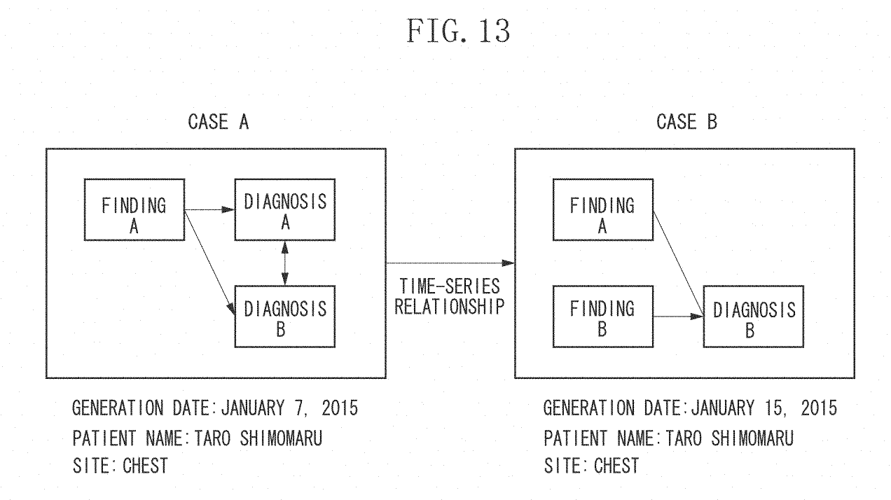

The cases of the search results can have a time-series relationship as illustrated in FIG. 13. In such a case, in order to distinguish the cases having a time-series relationship as a group, for example, the case presentation unit 122 displays the cases next to each other at the top of the list on the output device 15. This enables the doctor to find out with ease a path to follow in order to confirm the diagnosis based on the current report status. In the example illustrated in FIG. 12, a search using the search condition B and a search using the search condition C are sequentially executed, so two radiological interpretation reports generated at different times and dates in FIG. 13 are acquired. In such a case, the case presentation unit 122 displays the two radiological interpretation reports next to each other on the output device 15 so that the cases having a time-series relationship are easy to be recognized. While the GUIs illustrated in FIGS. 8A to 8D are not displayed visibly to the user in the present exemplary embodiment, this is not seen to be limiting, and the present exemplary embodiment can be combined with the first exemplary embodiment. Specifically, the search condition can be manually and automatically set.

### Modified Example 1

While the case search unit 121 is included in the report generation apparatus 10 in the first and second exemplary embodiments, this is not seen to be limiting. For example, the case search unit 121 can be provided outside the report generation apparatus 10. For example, the case search unit 121 can be provided to the HIS 20 including the radiological interpretation report database 2. If the radiological interpretation report database 2 is situated in the cloud, the case search unit 121 can be provided in the cloud.

In the case in which the case search unit 121 is provided outside the report generation apparatus 10, the report generation apparatus 10 includes a search condition acquisition unit (not illustrated) which acquires a search condition described above in the first and second exemplary embodiments. The search condition acquisition unit transmits the acquired search condition to the case search unit 121. The search condition to be transmitted can include, for example, the contents of the plurality of diagnosis entries and link information about links between the plurality of diagnosis entries. Specifically, the search condition acquisition unit corresponds to an example of a transmission unit configured to transmit as a search key a first diagnosis item including a description about a diagnosis, a second diagnosis item including a description about a diagnosis which is different from the first diagnosis item, and link information indicating a relationship between the first diagnosis item and the second diagnosis item.

As described above, the case search unit 121 provided outside the report generation apparatus 10 acquires from the radiological interpretation report database 2 a radiological interpretation report corresponding to the search condition as a search result and transmits the search result to the report generation apparatus 10. The search result includes the radiological interpretation report including the contents of a plurality of diagnosis entries specified in the search condition and link information about links between the plurality of diagnosis entries. The case presentation unit 122 receives the search result and displays the search result on the output device 15. Specifically, the case presentation unit 122 corresponds to an example of a reception unit configured to receive as a search result a diagnosis report which includes the first diagnosis item and the second diagnosis item and in which link information is added.

Specifically, the case search unit 121 needs to be situated anywhere within the medical information system illustrated in FIG. 2, and the location of the case search unit 121 is not limited to the location specified in the example illustrated in FIG. 5.

### Modified Example 2

While the case in which the case search unit 121 performs a case search using link information about links between diagnosis entries has been described in the first and second exemplary embodiments, this is not seen to be limiting. For example, the case search unit 121 can perform a case search using link information about links between finding entries including a description about the subject. Specifically, the case search unit 121 can execute a case search using link information about links between entries of the same category.

The present disclosure can also be realized by a circuit that realizes one or more functions (e.g., ASIC).

The information processing apparatus according to each of the above-described exemplary embodiments can be realized as a single apparatus, or a plurality of apparatuses can be communicably combined to execute the above-described process, both of which are encompassed in the exemplary embodiments. The above-described process can be executed on a shared server apparatus or servers. The plurality of apparatuses included in the information processing apparatus and the information processing system only needs to be capable of communicating at a predetermined communication rate and, further, does not have to be situated within the same institution or country.

Any combination of the above-described exemplary embodiments is also encompassed within the exemplary embodiments.

### Other Embodiments

Embodiment(s) can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been described, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-256152, filed Dec. 28, 2016, which is hereby incorporated by reference herein in its entirety.

What is claimed is:

1. An information processing apparatus configured to output a specific report from among a plurality of diagnosis reports, the information processing apparatus comprising:
an identification unit configured to identify, in a case where a first diagnosis item including a description about a diagnosis, a second diagnosis item including a description about a diagnosis that is different from the first diagnosis item, and link information indicating a relationship between the first diagnosis item and the second diagnosis item are acquired as a search key, a diagnosis report that includes the first diagnosis item and the second diagnosis item and in which the link information is added, from among a plurality of stored diagnosis reports; and
an output unit configured to output the identified diagnosis report.

2. The information processing apparatus according to claim 1, wherein in a case where information indicating a category of the link information is included in the search key, the identification unit identifies a diagnosis report that includes the first diagnosis item and the second diagnosis item and includes the link information of a same category as the category included in the search key.

3. The information processing apparatus according to claim 2, wherein the information includes information indicating that a relationship between a content described in the first diagnosis item and a content described in the second diagnosis item is a contradictory relationship, cause-and-effect relationship, or correspondence relationship.

4. The information processing apparatus according to claim 1, wherein in a case where the search key does not include the link information, the identification unit identifies a diagnosis report that includes the first diagnosis item and the second diagnosis item and does not include the link information.

5. The information processing apparatus according to claim 1, wherein in a case where the search key does not include the link information, the identification unit identifies a diagnosis report that includes the first diagnosis item and the second diagnosis item and does not include the link information and a diagnosis report that includes the first diagnosis item and the second diagnosis item and includes the link information.

6. The information processing apparatus according to claim 1, wherein the identified diagnosis report output by the output unit includes one or more of a first finding item corresponding to the first diagnosis item, a second finding item corresponding to the second diagnosis item, a first image corresponding to the first finding item, and a second image corresponding to the second finding item.

7. An information processing apparatus comprising:
a transmission unit configured to transmit, as a search key, a first diagnosis item including a description about a diagnosis, a second diagnosis item including a description about a diagnosis that is different from the first diagnosis item, and link information indicating a relationship between the first diagnosis item and the second diagnosis item; and
a reception unit configured to receive, as a search result based on the search key, a diagnosis report that includes the first diagnosis item and the second diagnosis item and in which link information is added.

8. A system comprising:
a transmission unit; and
an information processing apparatus configured to output a specific report from among a plurality of diagnosis reports,
wherein the information processing apparatus comprises:
an identification unit configured to identify, in a case where a first diagnosis item including a description about a diagnosis, a second diagnosis item including a description about a diagnosis that is different from the first diagnosis item, and link information indicating a relationship between the first diagnosis item and the second diagnosis item are acquired as a search key, a diagnosis report that includes the first diagnosis item and the second diagnosis item and in which the link information is added, from among a plurality of stored diagnosis reports, and
an output unit configured to output the identified diagnosis report, and
wherein the transmission unit transmits the search key to the information processing apparatus.

9. An information processing method configured to output a specific report from a plurality of diagnosis reports, the information processing method comprising:
identifying, in a case where a first diagnosis item including a description about a diagnosis, a second diagnosis item including a description about a diagnosis that is different from the first diagnosis item, and link information indicating a relationship between the first diagnosis item and the second diagnosis item are acquired as a search key, a diagnosis report that includes the first diagnosis item and the second diagnosis item and in which the link information is added from a plurality of stored diagnosis reports; and
outputting the identified diagnosis report.

10. A non-transitory computer readable storage medium storing a program for causing a computer to execute an information processing method configured to output a specific report from a plurality of diagnosis reports, the information processing method comprising:
identifying, in a case where a first diagnosis item including a description about a diagnosis, a second diagnosis item including a description about a diagnosis that is different from the first diagnosis item, and link information indicating a relationship between the first diagnosis item and the second diagnosis item are acquired as a search key, a diagnosis report that includes the first diagnosis item and the second diagnosis item and in which the link information is added from a plurality of stored diagnosis reports; and
outputting the identified diagnosis report.

11. An information processing apparatus configured to output a specific report from among a plurality of diagnosis reports, the information processing apparatus comprising:
an identification unit configured to identify, in a case where a first item including a description about a subject, a second item that is an item of a same category as a category of the first item and includes a description about the subject that is different from the first item, and link information indicating a relationship between the first item and the second item are acquired as a search key, a diagnosis report that includes the first item and the second item and in which the link information is added, from among a plurality of stored diagnosis reports; and
an output unit configured to output the identified diagnosis report.
